(19) United States
(12) Patent Application Publication
(10) Pub. No.: US 2022/0042099 A1
(43) Pub. Date: Feb. 10, 2022

(54) MOLECULAR AND FUNCTIONAL CHARACTERIZATION OF EARLY-STAGE PARKINSON'S DISEASE AND TREATMENTS THEREIN

(71) Applicants: QUADRANT BIOSCIENCES INC., Syracuse, NY (US); THE RESEARCH FOUNDATION FOR THE STATE UNIVERSITY OF NEW YORK, Syracuse, NY (US)

(72) Inventors: Frank A. MIDDLETON, Fayetteville, NY (US); Richard UHLIG, Ithaca, NY (US)

(73) Assignees: QUADRANT BIOSCIENCES INC., Syracuse, NY (US); THE RESEARCH FOUNDATION FOR THE STATE UNIVERSITY OF NEW YORK, Syracuse, NY (US)

(21) Appl. No.: 17/286,254

(22) PCT Filed: Oct. 18, 2019
(86) PCT No.: PCT/US2019/056884

§ 371 Date: Apr. 16, 2021

Related U.S. Application Data
(60) Provisional application No. 62/747,383, filed on Oct. 18, 2018.

(51) Int. Cl. C12Q 1/6883

(52) U.S. Cl. CPC C12Q 1/6883; C12Q 2600/178

(57) ABSTRACT

A method and composition for detecting, diagnosing, prognosing or monitoring a subject suspected of having or having Parkinson's disease by detecting miRNAs and microbial RNAs in saliva.

### CROSS-REFERENCE TO RELATED APPLICATION

This application claims the benefit of U.S. provisional application No. 62/747,383 filed Oct. 18, 2018.

The contents of U.S. Provisional 62/607,792, filed Dec. 19, 2017 AND U.S. Provisional 62/622,319 filed Jan. 26, 2018 are hereby incorporated by reference in their entirety.

### BACKGROUND OF THE INVENTION

Field of the Invention Early diagnosis, prognosis, and monitoring of Parkinson's disease (“PD”). Treatment regimens based on these diagnostic methods and on modulation of miRNA associated with Parkinson's disease.

Description of Related Art Parkinson's disease is a long-term degenerative disorder of the central nervous system that mainly affects the motor system.^{[1]} The symptoms generally come on slowly over time.^{[1]} Early in the disease, the prominent symptoms are shaking, rigidity, slowness of movement, and difficulty with walking.^{[1]} Thinking and behavioral problems may also occur and dementia becomes common in the advanced stages of the disease.^{[2]} Depression and anxiety are also common occurring in more than a third of people with PD.^{[2]} Other symptoms include sensory, sleep, and emotional problems.^{[1][2]} The main motor symptoms are collectively called “parkinsonism”, or a “parkinsonian syndrome”.^{[4]} The cause of Parkinson's disease is generally unknown, but believed to involve both genetic and environmental factors.^{[4]}

Parkinson's disease typically occurs in people over the age of 60, of which about one percent are affected.^{[1][3]} Males are more often affected than females.^{[4]} When it is seen in people before the age of 50, it is called young-onset PD. The average life expectancy following diagnosis is between 7 and 14 years.^{[2]}

The cause of Parkinson's disease is generally unknown, but believed to involve both genetic and environmental factors.^{[4]} Those with a family member affected are more likely to get the disease themselves.^{[4]} There is also an increased risk in people exposed to certain pesticides and among those who have had prior head injuries, while there is a reduced risk in tobacco smokers and those who drink coffee or tea.^{[4]} The motor symptoms of the disease result from the death of cells in the substantia nigra, a region of the midbrain resulting in an insufficient dopamine level^{[1]}. The reason for this cell death is poorly understood, but corresponds to the build-up of proteins into Lewy bodies in the neurons.^{[4]} Diagnosis of typical cases is mainly based on symptoms, with tests such as neuroimaging being used to rule out other diseases.^{[1]}

There is no cure for Parkinson's disease so treatment is directed at improving symptoms. Initial treatment is typically with the antiparkinson medication levodopa (L-DOPA), with dopamine agonists being used once levodopa becomes less effective.^{[2]} As the disease progresses and neurons continue to be lost, these medications become less effective while at the same time they produce a complication marked by involuntary writhing movements.^{[2]} Diet and some forms of rehabilitation have shown some effectiveness at improving symptoms. Surgery to place microelectrodes for deep brain stimulation has been used to reduce motor symptoms in severe cases where drugs are ineffective.[^{1]} Evidence for treatments for the non-movement-related symptoms of PD, such as sleep disturbances and emotional problems, is less strong.^{[4]}

Early diagnosis of Parkinson's disease as well as factors contributing to its development and progression would be desirable because it would permit earlier and targeted therapeutic intervention. However, discovery of reliable detection of markers for neurodegenerative diseases have been complicated by the inaccessibility of the diseased tissue such as the inability or risk to biopsy or test tissue from the central nervous system directly. Prior attempts have been made to profile mi-RNA (micro-RNA) in serum or cerebrospinal fluid (“CSF”) to associate particular markers with PD^{[5]}. And prior work has looked at a possible role of a brain-gut-microbiota axis dysregulation in Parkinson's disease^{[6]}. Another study focused on the identification of a protein marker (salivary protein DJ-1) as a potential PD biomarker^{[7]}. In view of the need for reliable, sensitive, specific, comparable, and accessible markers, or panels of markers, that correlate with PD as well as its etiology and/or symptoms, the inventors investigated correlations between miRNA and microbial RNAs in saliva.

### BRIEF SUMMARY OF THE INVENTION

A method for detecting, diagnosing, prognosing, or monitoring Parkinson's Disease comprising detecting abnormal levels of one or more miRNA and/or microbial RNAs in the saliva of a subject, and optionally, when an abnormal level is detected, further evaluating or testing the patient for Parkinson's Disease or treating the subject for Parkinson's Disease.

### BRIEF DESCRIPTION OF THE DRAWING(S)

FIG. 1. No differences in family, genus or species biodiversity measures in early stage PD. Whisker box plots indicate mean and range of Shannon alpha diversity (upper) and Bray-Curtis dissimilarity measures (lower) for the family, genus, and species levels of classification.

FIG. 2. ROC curve performance using the oral microbiome. The empirical ROC performance during cross-validation and its 95th percentile confidence interval are shown. Overall accuracy was 84.5%.

FIG. 3. Metabolic pathway changes in oral microbiome of early stage PD.

FIG. 4. Mixed Host+Taxa Model using up to all 31 total classifiers. AUC 0.941 100-fold Monte-Carlo CV. 86.9% Accuracy. Misclassified subjects 5/36 (control), 6/48 (PD).

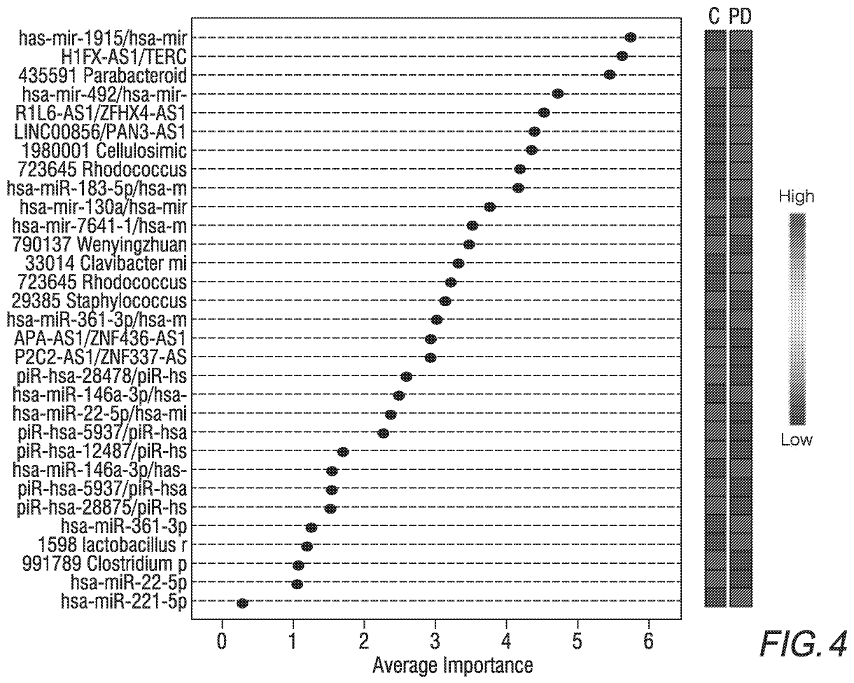

### DETAILED DESCRIPTION OF THE INVENTION

Given the complexity of PD, the difficulty in diagnosis and the inaccessibility of the nervous tissue, particularly the brain, to repeated sampling; the development of accessible biomarkers which can serve as indicators or sensors of the underlying pathophysiological processes is needed. From the extensive motor, cognitive, psychiatric, and autonomic symptoms, it is clear that multiple brain regions and peripheral tissues are affected in PD. Thus, the possibility that certain alterations in protein biomarkers can be used to diagnose PD was investigated.

The identification of biochemical markers that are easily accessible and accurately measured would represent a great advance in the diagnosis and treatment of PD. Since biological fluids such as blood and saliva are the most accessible and routine physical source of biological material available for diagnostic testing, saliva was mined for biomarkers for PD diagnostic development and testing.

Embodiments of the invention assessed the prevelance of numerous markers found in saliva as biomarkers for PD.

Saliva is a slightly alkaline secretion of water, mucin, protein, salts, and often a starch-splitting enzyme (as ptyalin) that is secreted into the mouth by salivary glands, lubricates ingested food, and often begins the breakdown of starches. Saliva is released by the submandibular gland, parotid gland, and/or sublingual glands and saliva release may be stimulated by the sympathetic and/or parasympathetic nervous system activity. Saliva released primarily by sympathetic or parasympathetic induction may be used to isolate microRNAs.

Saliva may be collected by expectoration, swabbing the mouth, passive drool, or by other methods known in the art. In some embodiments it may be withdrawn from a salivary gland. In some embodiments, a saliva sample may be further purified, for example, by centrifugation or filtration. For example, it may be filtered through a 0.22 micron or 0.45 micron membrane, and all membrane sizes in between, and the separated components used to recover microRNAs. In other embodiments, proteins or enzymes that degrade microRNA may be removed, inactivated or neutralized in a saliva sample.

Some representative, but not limiting saliva collection and miRNA purification procedures include purifying salivary RNA in accordance with, for example, the Oragene RNA purification protocol using TRI Reagent LS, a TriZol purification method, or similar method. The Oragene purification protocol generally includes multiple parts. In the first part, a sample is shaken vigorously for 8 seconds or longer and the sample is incubated in the original vial at 50° C. for one hour in a water bath or for two hours in an air incubator. In the second part, a 250-500 μL aliquot of saliva is transferred to a microcentrifuge tube, the microcentrifuge tube is incubated at 90° C. for 15 minutes and cooled to room temperature, the microcentrifuge tube is incubated on ice for 10 minutes, the saliva sample is centrifuged at maximum speed (>13,000×g) for 3 minutes, the clear supernatant is transferred into a fresh microcentrifuge tube and the precipitate is discarded, two volumes of cold 95% EtOH is added to the clear supernatant and mixed, the supernatant mixture is incubated at −20° C. for 30 minutes, the microcentrifuge tube is centrifuged at maximum speed, the precipitate is collected while the supernatant is discarded, the precipitate is dissolved in 350 μL of buffer RLT, and 350 μL of 70% EtOH is added to the dissolved pellet mixture and mixed by vortexing. The first two parts may be followed by the Qiagen RNeasy cleanup procedure.

The purification process may further include a second purification step of, for example, purifying the saliva sample using a RNeasy mini spin column by Qiagen. The purification of a biological sample may include any suitable number of steps in any suitable order. Purification processes may also differ based on the type of a biological sample collected from the subject. The yield and quality of the purified biological sample may be assessed via a device such as an Agilent Bioanalyzer, for example, to determine if the yield and quality of RNA is above a predetermined threshold.

microRNA or miRNA is a small non-coding RNA molecule containing about 22 nucleotides, which is found in plants, animals and some viruses, that functions in RNA silencing and post-transcriptional regulation of gene expression (see Ambros et al., 2004; Bartel et al., 2004). MicroRNAs affect expression of the majority of human genes, including CLOCK, BMAL1, and other circadian genes. Notably, miRNAs are released by cells that make them and circulate throughout the body in all extracellular fluids where they interact with other tissues and cells. Recent evidence has shown that human miRNAs even interact with the population of bacterial cells that inhabit the lower gastrointestinal tract, termed the gut microbiome. Moreover, circadian changes in the gut microbiome have recently been established. Small non-coding RNAs (miRNAs) suppress protein expression and that have emerged as useful biomarkers in cancer, diabetes, neurodevelopmental, and neurodegenerative disorders. Although miRNAs are made in all tissues and organs of the body, many of them show tissue-specificity. Moreover, miRNAs can act within the cells that synthesize them or be released into the extracellular space (EC) and travel in biofluids to affect other cells. Numerous studies have shown that miRNA expression profiles differ between healthy and diseased states, and that the release of miRNAs into the EC appears elevated following tissue damage. Epigenetic data includes data about miRNAs. Among the objectives of the inventors were to establish the relationship between peripheral measures of miRNA, objective assessment of likely mTBI severity, and sensitive indices of balance and cognitive function.

A miRNA standard nomenclature system uses the prefix “miR” followed by a dash and a number, the latter often indicating order of naming. For example, miR-120 was named and likely discovered prior to miR-241. A capitalized “miR-” refers to the mature form of the miRNA, while the uncapitalized “mir-” refers to the pre-miRNA and the pri-miRNA, and “MIR” refers to the gene that encodes them. The prefix “hsa-” denotes a human miRNA.

The sequences of miRNAs are known and may be obtained by reference to MirBase, Hyper Text Transfer Protocol (HTTP)://WorldWideWeb.mirbase.org/blog/2018/03/mirbase-22-release/ (last accessed Mar. 19, 2018, incorporated by reference) and/or to Hyper Text Transfer Protocol (HTTP)://WorldWideWeb.mirbase.org/index.shtml (last accessed Mar. 19, 2018; incorporated by reference).

miRNA elements. Extracellular transport of miRNA via exosomes and other microvesicles and lipophilic carriers is an established epigenetic mechanism for cells to alter gene expression in nearby and distant cells. The microvesicles and carriers are extruded into the extracellular space, where they can dock and enter cells, and block the translation of mRNA into proteins (Hu et al., 2012). In addition, the microvesicles and carriers are present in various bodily fluids, such as blood and saliva (Gallo et al., 2012), enabling us to measure epigenetic material that may have originated from the central nervous system (CNS) simply by collecting saliva. In fact, the inventors believe that many of the detected miRNAs in saliva are secreted into the oral cavity via sensory nerve afferent terminals and motor nerve efferent terminals that innervate the tongue and salivary glands and thereby provide a relatively direct window to assay miRNAs which might be dysregulated in the CNS of individuals. Thus, extracellular miRNA quantification in saliva provides an attractive and minimally-invasive technique for brain-related biomarker identification in children with a disease or disorder or injury. Moreover, this method minimizes many of the limitations associated with analysis of post-mortem brain tissue or peripheral leukocytes (relevance of expression changes, painful blood draws) employed previously.

miRNA isolation from biological samples such as saliva and their analysis may be performed by methods known in the art, including the methods described by Yoshizawa, et al., Salivary MicroRNAs and Oral Cancer Detection, Methods Mol. Biol., 2013; 936: 313-324 or by using commercially available kits, such as mirVana™ miRNA Isolation Kit).

The microbiome of the gastrointestinal (GI) tract is essential for mammalian physiology, aiding digestion, synthesis, and absorption of important nutritional components such as amino acids, folate, and B vitamins. Accumulating evidence suggests that the GI microbiome also influences host behavior and neurodevelopment through the “microbial-gut-brain axis”. This axis represents an evolving concept of microbial-mediated cross-talk between the central nervous system (CNS) and GI tract that occurs through several different modalities, including direct neural activation, immune modulation, and hormonal, peptidergic, and epigenetic signaling.

microbiome elements. Based on the studies described herein, the inventors have hypothesized that components of the oral microbiome may correlate with the diagnosis of Parkinson's Disease and/or specific behavioral symptoms. As the microbiome can be simultaneously detected using our salivary RNA diagnostic technology, the inventors have evaluated whether inclusion of components of the microbiome would improve diagnostic accuracy for Parkinson's disease. This ability to jointly monitor the miRNA and microbiome elements of the microtranscriptome gains additional significance in view of recent data that miRNA levels can strongly fluctuate in concert with the host microbiome. Moreover, other recent studies have revealed that alterations in the gut microbiome can affect the expression of brain miRNAs in mice, along with the production of anxiety symptoms. Thus, the interaction of the host miRNA elements and the GI microbiome elements and their joint effects on the brain and behavior comprises a key component of our current biomarker discovery path.

Some of the microorganisms as part of the microbiome that can be assessed include at least one of those listed in Table A below:

TABLE A
Bacteria families altered in early stage PD in the present study and in prior PD studies.

| | Saliva | | | |
|---|---|---|---|---|
| Microbe Family | Change | Family Member Changes | Previous Studies | Involved In |
| Lactobacillaceae | Increase | Lactobacillus | Family | An increase in L. reuteri |
| | (↑) | Lactobacillus acidophilus | Pereirao^{a,b} (↑) | lead to greater activity of |
| | | Lactobacillus fermentum | Hills-Burns^{c} (↑) | ENS neurons and vagal |
| | | Lactobacillus plantarum | Hopfner^{c} (↑) | afferents, can lead to |
| | | Lactobacillus reuteri | Scheparjans^{c} (↑) | increased secretion of |
| | | Lactobacillus salivarius | Bedarf^{c} (↓) | alpha synuclein (Perez- |
| | | | | Burgos et al., 2013; Kunze |
| | | Other Studies | Genus Lactobacillus | et al., 2009; Paillusson et |
| | | Lactobacillus mucosae (↑) | Hills-Burns^{c} (↑) | al., 2012) |
| | | | Hasegawa^{c} (↑) | |
| | | | Petrov^{c} (↑) | Increased Lactobacillaceae |
| | | | Unger^{c} (↓) | levels are associated with |
| | | | | decreased ghrelin levels |
| | | | Species/OTU | (Scheperjans et al., 2015; |
| | | | Petrov^{c} (↑) | Unger et al., 2011). |
| | | | L. mucosae | |
| | | | | Lactobacillus produces |
| | | | | GABA and acetylcholine |
| | | | | (Cryan and Dinan, 2012) |
| | | | | L. reuteri reduces anxiety |
| | | | | and corticosterone in mice |
| | | | | (Bravo et al., 2011) |
| | | | | Lactobacilli species |
| | | | | beneficial in treatment of |
| | | | | constipation, diarrhea, and |
| | | | | IBS symptoms (Fijan, |
| | | | | 2014) |
| Bifidobacteriaceae | Increase | Bifidobacterium | Family | Bifidobacteria have anti- |
| | (↑) | Bifidobacterium animalis | Hills-Burns^{c} (↑) | inflammatory properties |
| | | Bifidobacterium dentium | Bedarf^{c} (↑) | (Mulak and Bonaz, 2015). |
| | | Bifidobacterium longum | Hopfner^{c} (↑) | |
| | | | Keshararzi^{d} (-) | Bifidobacteria affect local |
| | | Gardnerella | | and system immune |
| | | Gardnerella vaginalis | Genus | responses (Ventura et al., |
| | | Parascardovia | Bifidobacterium | 2014). |
| | | Parascardovia denticolens | Unger^{c} (↑) | |
| | | Scardovia | Hills-Burns^{c} (↑) | Bifidobacterium produce |
| | | | Petrov^{c} (↑) | GABA (Cryan and Dinan, |
| | | Other Studies | Keshararzi^{d} (-) | 2012) |
| | | OTU4347159 | Hasegawa^{c} (-) | |
| | | (Bifidobacterium) (↑) | | B. longum reduces anxious |
| | | | Species/OTU | behavior in animals and |
| | | | Hills-Burns^{c} (↑) | decreased serum cortisol |
| | | | OTU4347159 | in humans (Messaoudi et |
| | | | (Bifidobacterium) | al., 2011) |
| | | | | Bifidobacterium used for |
| | | | | treatment for constipation, |
| | | | | diarrhea, IBS symptoms, |
| | | | | and GI disorders (Fijan, |
| | | | | 2014) |
| | | | | Gardnerella vaginalis is |
| | | | | associated with bacterial |
| | | | | vaginosis (Chang et al., |
| | | | | 2003). |
| | | | | Parascardovia denticolens |
| | | | | is found in dental caries |
| | | | | (Oshima et al., 2015) |
| Saccharomycetaceae | Increase | Candida | Species/OTU | Anecdotal associations of |
| | (↑) | Candida alicans | Hills-Burns^{c} (↑) (↓) | Candida with PD |
| | | Candida duliniensis | OTU4439469(Torulas | symptoms |
| | | Sacccaromyces cerevisiae | pora) (↑) | |
| | | Torulaspora | OTU180999 | Candida produces |
| | | Torulaspora delbrueckii | (Torulaspora) (↓) | serotonin (Cryan and |
| | | | OTU4325096 | Dinan, 2012) |
| | | Other Studies | (Torulaspora) (↓) | |
| | | OTU180999 (Torulaspora) | OTU4457438 | Sacccaromyces cerevisiae |
| | | (↓) OTU4325096 | (Torulaspora) (↓) | produces Ndi 1p which can |
| | | (Torulaspora) (↓) | | restore function in mice |
| | | OTU4457438 (Torulaspora) | | ETC complex 1 that is lost |
| | | (↓) OTU4439469 | | due to Pink 1 mutations |
| | | (Torulaspora) (↑) | | (Vilain et al., 2012) |
| Acidaminococcaceae | Increase | Current and Previous Studies | Family | Acidaminococcus |
| | (↑) | Acidaminococcus | Bedarf^{c} (↑) | consumes glutamate which |
| | | | | is important for oxidation |
| | | | Genus | in the intestinal epithelium |
| | | | Acidaminococcus | (Gough et al., 2015). |
| | | | Li^{c} (↑), | |
| Vibrionaceae | Increase | Lucibacterium_sp_LPB0138 | None | |
| | (↑) | | | |
| Brucellaceae | Increase | Brucella | None | Brucella is the cause of |
| | (↑) | | | Brucellosis (Alton and |
| | | | | Forsyth, 1996) |
| Methylobacteriaceae | Increase | Methylobacterium | None | |
| | (↑) | | | |
| Nocardiaceae | Increase | Rhodococcus | None | Rhodococcus aurantiacus |
| | (↑) | Rhodococcus_sp_008 | | induces encephalitis in |
| | | | | mice and causes |
| | | | | movement disorders due to |
| | | | | inflammation mediated by |
| | | | | T cells; motor symptoms |
| | | | | improve with L-Dopa |
| | | | | (Min et al., 1999) |
| Microbacteriaceae | Increase | Clavibacter, | None | |
| | (↑) | Clavibacter michiganensis | | |
| Promicromonosporaceae | Increase | Cellulosimicrobium | None | Cellulomicrobium_sp_TH_20 |
| | (↑) | Cellulomicrobium_sp_TH_20 | | transforms |
| | | | | ginsenosides which have |
| | | | | anti-inflammatory |
| | | | | properties (Yu et al., 2017) |
| Enterobacteriaceae | Decrease | Buchnera, | Family | Escherichia produces |
| | (↓) | Buchnera aphidicola, | Unger^{c} (↑) | noradrenaline and |
| | | | Bedarf^{c} (↑) | serotonin (Cryan and |
| | | Other Studies | Keshararzia^{d} (-) | Dinan, 2012) |
| | | Escherichia (↓) | Hasegawa^{c} (-) | |
| | | | | Escherichia is a possible |
| | | | Genus Escherichia | treatment for constipation, |
| | | | Li^{c} (↑) | IBS, GI disorders, |
| | | | Keshararzia^{d} (-) cb, | ulcerative colitis, Crohn's |
| | | | | disease, and colon cancer |
| | | | | (Fijan, 2014) |
| | | | | Increase in |
| | | | | Enterobacteriaceae is |
| | | | | associated with postural |
| | | | | instability and gait |
| | | | | difficulty (PIGD) |
| | | | | phenotype (Scheperjans et |
| | | | | al., 2015) |
| Rhizobiaceae | Decrease | Candidatus azobacteroides | None | |
| | (↓) | Candidatus azobacteroides- | | |
| | | pseidotrichonymphae | | |
| Campylobacteraceae | Decrease | Campylobacter ureolyticus | None | Campylobacteraceae |
| | (↓) | | | implicated in acute GI |
| | | | | distress, diarrhea |
| | | | | (Lastovica et al., 2014) |
| Streptococceae | Bidirectional | Streptococcus inopinata (↑) | Family | S. mutans contributes to |
| | (↓) (↑) | Streptococcus mutans (↑) | Bedarf^{c} (↓) | tooth decay via production |
| | | Streptococcus_phage_PhiSpn | | of acidic metabolites |
| | | 200 (↓) | Genus Streptococcus | (Loesche, 1986) |
| | | Streptococcus_sp_I_G2 (↓) | Li^{c} (↑) | |
| | | | | Streptococcus produces |
| | | Other Studies | | serotonin (Cryan and |
| | | Streptococcus (↑) | | Dinan, 2012) |
| Bacillaceae | Bidirectional | Baccillus megaterium (↓) | Family | Bacillus produces |
| | (↓) (↑) | Bacillus_sp_FJAT_2290 (↑) | Hopfner^{c} (+75) | noradrenaline and |
| | | Halobacillus mangrove (↓) | | dopamine (Cryan and |
| | | | Species/OTU | Dinan, 2012) |
| | | Other Studies | Hopfner^{c} (↓) | |
| | | Incertae sedis XII (↓) | Incertae sedis XII | Bacillus species reduce |
| | | | | diarrhea and prevent caries |
| | | | | (Fijan, 2014) |
| | | | | Bacillus sp JPJ produces |
| | | | | L-DOPA (Surwase and |
| | | | | Jadhav, 2011). |
| Flavobacteriaceae | Bidirectional | Capnocytophaga canimorsus | Family | Flavobacteriaceae have |
| | (↓) (↑) | (↑) | Pereirao^{a,b} (↓) | antioxidative properties |
| | | Chryseobacterium (↓) | Bedarf^{c} (↓) | (Choi and Choi, 2015) |
| | | Chryseobacterium_sp_IHB_17019 | | |
| | | (↓) | Species/OTU | |
| | | Wenyingzhuangia (↓) | Pereira^{a,b} (↓) | |
| | | Wenyingzhuangia fucanilytica | OTU000509 | |
| | | (↓) | (Capnocytophaga) (↓) | |
| | | Bacterium_3519_10 (↓) | OTU000123 (↓) | |
| | | Other Studies | | |
| | | OTU000509 | | |
| | | (Capnocytophaga) (↓) | | |
| | | OTU000123 (↓) | | |

Arrows indicate direction of microbiome changes in PD subjects; (-) represents insignificant change; Superscripts indicate tissue source: a, oral; b, nasal; c, fecal; d, colon biopsy

The inventors refined their technique for saliva collection and improved the software/statistical pipeline for RNA processing. As a result, it has become possible to measure both human and non-human RNA within a single sample. This approach has allowed the inventors to define a panel of miRNAs and microbial species that are differentiated in Parkinson's disease, as is described hereinafter. The methods disclosed by the inventors also comprise selecting a set miRNAs, and a set of microbial taxons that can be combined with appropriate weighting coefficients, or used in ratios, to generate a prediction of association with Parkinson's Disease.

Sex and several other biological factors of relevance are considered as potential modifiers of outcome for the utility of our diagnostic tools. Nevertheless, it is absolutely essential that any molecular diagnostic tool that has been developed by the inventors is equally accurate for both males and females. A broad range of clinical, biological and neuropsychological variables are collected at each site on all subjects and specifically examined in all statistical models. Such variables include age, sex, ethnicity, birth age, birth weight, perinatal complications, current weight, body mass index, current oropharyngeal status (allergic rhinitis, sinus infection, cold/flu, fever, dental carries), sleep disorders, gastrointestinal issues, diet, current medications, chronic medical issues, immunization status, medical allergies, dietary restrictions, early intervention services, hearing deficits, visual deficits, surgical history, and family psychiatric history. Rigorous neuropsychological evaluation of the subjects using standardized, age-appropriate and validated measures of Parkinson's indicia may also performed. The results of the inventors” molecular studies are directly compared with all of these in an unbiased manner to determine the specific magnitude of any interacting effects or to test for the presence of associations in the data that might be of interest. The inventors also used a set or a group of patient data to input to the algorithm.

During sleep-wake cycles there are numerous molecular, cellular, and physiological changes that occur. Many of these changes are driven by what are referred to as circadian regulatory genes, such as CLOCK and BMAL1. These, in turn, cause numerous changes in the expression of physiologically relevant genes, proteins, and hormones. Apart from light-dark cycles, the factors that influence expression of circadian genes are not fully understood. Taken together, the inventors' data suggest a previously unknown relationship between saliva miRNA and microbe content as well as temporal influences (i.e., temporal variations) on miRNAs (and/or microbes) themselves. The systems and methods described herein to normalize epigenetic data (sequencing data or other data) that experience temporal variations may be used in any suitable application where temporal variations may affect the data. Thus, in another aspect of the invention, the data set(s) may be normalized to account for temporal variations in sample prevalence. For instance, by determining read-counts of one or more miRNAs or other genetic markers in a biological sample taken from a subject, normalizing epigenetic data of the subject to account for inter-sample read-count variations, wherein the read-count normalization uses one or more invariant miRNAs, determining time of day that the biological sample was taken, and applying an algorithm to the read-count normalized miRNAs, wherein the algorithm uses the time-of-day to normalize the subject's miRNA expression levels relative to time-of-day.

One aspect of the invention is a kit suitable for determining whether a subject has a disease, disorder, or condition including 2 or more miRNA probes of a probe set. Each miRNA probe may include a ribonucleotide sequence corresponding to a specific miRNA described herein. In an implementation, the kit further may include a solid support attached to the 2 or more miRNA probes. In an implementation, the kit may further include at least one of the following: (a) one randomly generated miRNA sequence adapted to be used as a negative control; (b) at least one oligonucleotide sequence derived from a housekeeping gene, used as a standardized control for total RNA degradation; or (c) at least one randomly-generated sequence used as a positive control. Alternatively, a probe set may include miRNA probes having ribonucleotide sequences corresponding to DNA sequences from particular microbiomes described herein.

Another objective of the inventors was to provide a method a method of monitoring progression of a disorder, disease state or injury in a subject, comprising:

analyzing at least two biological samples from the subject taken at different time points to determine a read-count and time-of-day normalized expression levels of one or more specific miRNAs or other genetic markers in each of the at least two biological samples, and comparing the determined levels of the one or more specific miRNAs over time to determine if the subject's read-count and time-of-day normalized expression levels of the one or more specific miRNAs is changing over time, wherein an increase or decrease in the read-count and time-of-day normalized expression levels of the one or more specific miRNAs over time is indicative that the subject's disorder or disease state or injury is improving or deteriorating. In one embodiment, miRNAs subject to time-of-day normalization are selected from the group consisting of Group A circaMiRs and/or those miRNA which share the seed sequences of the Group A circaMiRs.

The analysis can be performed using linear regression analyses, statistical analyses and/or other computer based models of assessing large volumes of data with multiple variables, sometimes each variable being given different weights in the final scoring and/or conclusions based on the data set.

A key aspect of the invention is to identify those subjects at risk for and/or having Parkinson's disease so that a clinician can have more information on a disease that is difficult, at times, to diagnose, particularly in the early stages of the disease. The methodology described herein can be used by itself but preferably with other standard indicia of Parkinson's indicia, for instance, symptoms of Parkinsonism such as bradykinesia, hypokinesia and akinesia.

Thus, once a subject has been determined to be an at risk patient and/or having Parkinson's disease, the subject is treated to reduce and/or attenuate further progression of the disease. Various medications and treatments are known in the art and include Carbidopa-levodopa, Dopamine agonists such pramipexole, ropinirole, rotigotine, and apomorphine, MAO B inhibitors such as selegiline, rasagiline and safinamide, Catechol O-methyltransferase (COMT) inhibitors such as entacapone, anticholinergic medications such as benztropine and trihexyphenidyl, Amantadine, deep brain stimulation, other surgical interventions, prescribed diet and exercise programs

### Examples

### Methods

### Study Design

This was a cross-sectional case-control design employing high throughput RNA sequencing to examine salivary microbial RNAs in subjects with early stage Parkinson's disease and healthy age and gender matched controls.

### Subject Ascertainment

This study was approved by the Institutional Review Board for the Protection of Human Subjects (IRB) at SUNY Upstate Medical University in Syracuse, N.Y. Informed written consent was obtained for all human subjects. Subjects were recruited from the greater Syracuse and Upstate New York area and received copies of the study description, consent documentation, and a comprehensive health and symptom questionnaire packet prior to their study visit. The questionnaire packet encompassed a detailed medical and health history and six standardized instruments: (1) Part I of the Movement Disorder Society—Unified Parkinson's Disease Rating Scale (MDS-UPDRS-I/II) referred to as Non-Motor Aspects of Experiences of Daily Living, (2) Part II of the MDS-UPDRS, referred to as the Motor Experiences of Daily Living, (3) The Scales for Outcomes in Parkinson's Disease Autonomic questionnaire (SCOPA-AUT), (4) The Parkinson's Disease Quality of Life Scale (PDQUALIF), (5) The Non-Motor Symptom Questionnaire (NMS), and (6) The Beck Depression Inventory (BDI).

### Inclusion/Exclusion Criteria

Subjects included in the Parkinson's disease (PD) group had been previously diagnosed by a neurologist and met the general diagnostic criteria for late-onset PD, including bradykinesia, rigidity, and typically a resting tremor. Exclusion criteria included a history of neuroleptic use or moderate to severe TBI that might have contributed to trauma-induced parkinsonism. Control subjects were included if they had no prior history of major medical procedures or conditions, were never on PD medications or suspected of having a movement disorder, and did not have any first-degree relatives with PD.

### Functional Evaluation

All PD subjects were evaluated using Part III of the MDS-UPDRS by a movement disorder specialist or trained Ph.D.-level evaluator. PD subjects also completed a spiral tracing test and cursive handwriting test to screen for persistent non-resting tremor as well as micrographia, and underwent resting tremor measurements in both hands while wearing a highly sensitive accelerometer (sampling frequency=250 Hz). Height, weight, blood pressure and pulse were obtained on all subjects. All subjects then completed a detailed sensory, motor, cognitive, and balance assessment that included: (1) 12-item Modified Brief Smell Identification Test (mBSIT); (2) 10-item taste test (for sweet, salty, sour and bitter solutions); (3) Trailmaking A test; (4) Trailmaking B test; (5) Digit Span Forward test; (6) Digit Span Reverse test; (7) Simple Reaction Time (SRT); (8) Procedural Reaction Time test (PRT); (9) Go/No-Go test (GNG); and (10) balance/body sway measurements (30 seconds duration) with their shoes off in 10 different postures, while wearing an accelerometer around their waste. With the exception of the two sensory measures, these items were part of ClearEdge®, an integrated FDA-listed tablet-based functional assessment system (Quadrant Biosciences, Inc.) that incorporates three simple and complex reaction time measures (SRT, PRT, GNG) from DANA BrainVitals® battery (AnthoTronix, Inc.) along with the measurements of postural sway and cognitive performance. The postures that were used were as follows: Two legs side by side, eyes open, on a hard surface (TLEO); Two legs side by side, eyes closed, on a hard surface (TLEC); Tandem stance, eyes open, on a hard surface (TSEO); Tandem stance, eyes closed, on a hard surface (TSEC); Two legs side by side, eyes open, on a foam pad (TLEOFP); Two legs side by side, eyes closed, on a foam pad (TLECFP); Tandem stance, eyes open, on a foam pad (TSEOFP); Tandem stance, eyes closed, on a foam pad (TSECFP); a simple dual task involving tandem stance, eyes open, on a hard surface while holding the tablet device (TSEOHT); and a complex dual task involving completion of Trailmaking B while holding the tablet, with two legs side by side, eyes open, on a hard surface (TLEOCT).

Raw demographic data were compiled for all subjects. The functional Balance, Motor, and Cognitive score data were converted to z scores by direct comparison of each subject to a trimmed set of data that represented the mean of the control group after removal of any outlier data points (exceeding+/−2 standard deviations) from any of the measures in the control data set. The outlier points were retained however, for the between group comparisons. The resulting set of 35 demographic and functional variables were then screened for normality in PD and controls separately using the Shapiro-Wilk Test. This indicated that more than half of the variables in both subject groups failed the normality test. Accordingly, we used a Mann-Whitney test, with false discovery rate set at FDR<0.1 to identify rank-based differences between PD and control groups. For simplicity, however, all demographic and functional differences reported are either mean percentage or z score differences. The additional clinical and functional data obtained on the PD subjects was compiled as relative frequencies or raw values and instrument scores. These values were cross-referenced where appropriate to established cutoff values for mild to moderate PD symptom severity based on published literature.

### Saliva Collection and Processing

Subjects provided a saliva sample by expectoration into an OraGene RNA (RE-100) collection vial (DNA Genotek, Ottawa, ON). At least 30 minutes had elapsed between the time of last food or drink consumption and saliva collection. Before collecting saliva samples, each subject rinsed their mouth with bottled water. Approximately 1 mL of saliva was obtained from each participant. Samples were stored at room temperature during the study visit and then at 4 C until processing. A Trizol method was used to purify the salivary RNA and a second round of purification was followed using an RNEasy mini column (Qiagen). Yield and quality of the RNA samples was assessed with the RNA NanoChip on the Agilent Bioanalyzer prior to library construction using the Illumina TruSeq Small RNA Sample Prep protocol (Illumina; San Diego, Calif.). Identification and quantification of microbial RNA was performed using next generation sequencing (NGS) on a NextSeq 500 instrument (Illumina). Sets of 48 samples were indexed together at a targeted depth of 10 million single-end 75 bp reads per sample. De-indexing, adapter trimming and quality control metrics were obtained from Partek Flow software. Alignment of microbial transcripts was performed using the K-Slam software, which references the NCBI Taxonomy database, after filtering to remove miRNAs and other RNAs that aligned to the human transcriptome. Taxons were defined by their family, genus, species, and subspecies (when available).

The microbial RNA present in raw counts of 10 or more in at least 10% of samples were interrogated for differences between subject groups in overall richness using the Shannon alpha diversity and Bray-Curtiss beta diversity metrics. The set of genus and species data were then examined for between group differences using the Mann-Whitney test with false discovery correction (FDR<0.05) and for the ability to completely distinguish the subjects in a binomial classification test using logistic regression with receiver cperating characteristic (ROC) curve analysis (with 10-fold cross-validation). The biological significance of differential microbial transcript abundance was assessed using KEGG Pathway mapping as well as hierarchical clustering analysis within MicrobiomeAnalyst and MetaboAnalyst R packages. Correlations between different microbial and functional and demographic measures were assessed in an exploratory manner by Pearson product-moment correlation analysis.

### Results

### Participants

A total of 84 subjects completed the study, including 36 healthy controls with no history of movement disorder and 48 subjects with early stage PD (Table 1). None of the participants had active dental caries or periodontal disease.

TABLE 1
Subject Demographics

| | % | | | | | Systolic | | Ave |
|---|---|---|---|---|---|---|---|---|
| Group | Male | Age | Height | Weight | BMI | BP | Pulse | Sleep |
| Par- | 60.4 | 69.5 | 67.1” | 174.9 | 26.9 | 131.4 | *72.2 | 7.0 |
| kinson | | | | | | | | hrs |
| (n = 48) | | | | | | | | |
| Control | 55.6 | 68.5 | 67.1” | 168.7 | 26.2 | 130.3 | 66.6 | 7.5 |
| (n = 36) | | | | | | | | hrs |

*Significant (FDR <0.04) difference versus Control group

### Functional Outcomes

Among the PD subjects, the average duration of a diagnosis was 3.4 years (SE±0.56 years), with an average Hoehn & Yahr Stage of 1.92, and average scores for subscales of the MDS-UPDRS, NMS, SCOPA-AUT, PDQUALIF, and BDI all falling in established ‘mild’ ranges for those instruments according to published criteria (UPDRS-I 10.0, UPDRS-II 8.6, UPDRS-III 23.9) Most PD subjects (69%) were observed to have resting tremor and 87% were on PD medication (Table 2). Notably, more than 95% of our PD subjects had evidence of upper or lower GI disturbance (Table 2).

TABLE 2
PD Subject Characteristics

| Scale/Subscale | Average | Typical Cutoff - | Reference |
|---|---|---|---|
| UPDRS-I | 10.0 | 10-11 | PMID: 25466406 |
| UPDRS-II | 8.6 | 12-13 | PMID: 25466406 |
| UPDRS-III | 23.9 | 32-33 | PMID: 25466406 |
| Hoehn & Yahr Stage | 1.92 | 3.0 | PMID: 15372591 |
| Duration of illness | 3.44 | | |
| Noted Resting Tremor % | 68.8 | | |
| Anti-PD medication % | 87.5 | | |
| Sleep Dysfunction % | 83.3 | | |
| Oropharyngeal Dysfunction % | 85.4 | | |
| Thermoregulatory, | 90.0 | | |
| Vasomotor Dysfunction % | | | |
| GI or Urinary Dysfunction % | 95.8 | | |
| NMS Questionnaire | 8.0 | 8.8-12.0 | PMID: 17546669 |
| SCOPA-AUT | 12.0 | 16-17 | PMID: 15390007 |
| PDQUALIF | 35.25 | 37.7-38.8 | PMID: 12784266 |
| Beck Depression Inventory | 7.4 | 13 | PMID 13688369 |

Compared with healthy control subjects, the PD subjects in our cohort were found to exhibit significant changes in several indices of motor, cognitive and sensory function. Specifically, the early stage PD subjects showed a significant increase in completion time for the Trailmaking A and B tasks, and a decrease in Trailmaking B completion score (Table 3). These deficits were present in the absence of a significant change in Simple Reaction Time Score, although a trend for slower reaction times was apparent. Complementing these findings, the Procedural Reaction Time (PRT) Score was also significantly decreased in the DANA Brain Vitals set of measures (SRT, PRT, GNG) (Table 3).

TABLE 3
Motor, Cognitive, and Sensory Outcome Measures

| | | | z score diff | |
|---|---|---|---|---|
| Measure | PD | CTRL | (or % diff) | FDR |
| Trailmaking A (Completion Time) | 1.47 | 0.20 | 1.27 | 0.0034 |
| Trailmaking A (Completion Score) | −0.41 | 0.0 | −0.41 | 0.0768 |
| Trailmaking B (Completion Time) | 1.84 | 0.36 | 1.48 | 0.0653 |
| Trailmaking B (Completion Score) | −0.88 | 0.0 | −0.88 | 0.0024 |
| Digit Span Forward (Score) | −0.15 | 0.0 | −0.15 | 0.4513 |
| Digit Span Reverse (Score) | −0.08 | 0.0 | −0.08 | 04590 |
| Two Legs EO (TLEO Balance Score) | −1.80 | 0.0 | −1.80 | 0.0026 |
| Two Legs EC (TLEC Balance Score) | −0.39 | 0.0 | −0.39 | 0.0250 |
| Tandem Stance EO (TSEO Balance Score) | −0.87 | 0.0 | −0.87 | 0.0258 |
| Tandem Stance EC (TSEC Balance Score) | −0.30 | −0.25 | −0.05 | 0.4724 |
| Two Legs EO Foam Pad (TLEOFP Balance Score) | −0.38 | 0.0 | −0.38 | 0.0665 |
| Two Legs EC Foam Pad (TLECFP Balance Score) | −0.63 | −0.22 | −0.41 | 0.1468 |
| Tandem Stance EO Foam Pad (TSEOFP Balance Score) | −0.26 | −0.27 | 0.01 | 0.4824 |
| Tandem Stance EC Foam Pad (TSECFP Balance Score) | 0.55 | 0.0 | 0.55 | 0.0245 |
| Holding Tablet Dual Task (TSEOHT Balance Score) | −0.24 | 0.0 | −0.24 | 0.4196 |
| Trailmaking B_Dual Task (Balance Score) | −1.50 | −1.08 | −0.43 | 0.1578 |
| Trailmaking B_Dual Task (Completion Score) | −0.42 | 0.0 | −0.42 | 0.0463 |
| Trailmaking B Dual Task (Completion Time) | 1.65 | 0.34 | 1.31 | 0.0266 |
| Simple Reaction Time (SRT Score) | −0.60 | −0.12 | −0.48 | 0.0622 |
| Procedural Reaction Time (PRT Score) | −0.96 | −0.10 | −0.85 | 0.0082 |
| Go/NoGo (GNG Score) | −0.65 | −0.11 | −0.54 | 0.0265 |
| Taste test (Raw Score/10) | 6.81 | 8.2 | −17% | 0.0000 |
| Smell test (Raw Score/12) | 7.42 | 10.3 | −28% | 0.0000 |

Balance Scores. PD subjects were found to exhibit increased body sway (decreased score) in four of the balance measures (TLEO, TLEC, TSEO, TLEOFP) and an apparent increase in the performance of one balance measure (TSECFP) (Table 3). However, inspection of the data for this latter task, which is usually considered the most difficult, indicated that the higher scores were likely due to selection bias of more capable PD subjects, because multiple PD subjects (n=7) were actually unable to complete it. This was also true for another task that is considered nearly as difficult (TSEOFP), where 8 PD subjects were unable to complete it and there was no overall between group difference. Thus, discounting the TSECFP and TSEOFP tasks, the overall trend when the full group scores were available was for reduced balance scores in the PD group.

Reaction Times and Cognitive Scores. PD subjects demonstrated a consistent decrease in their simple and complex reaction time measures compared with Controls, as reflected in reduced performance on the SRT, PRT and GNG tasks (Table 3). Consistent with the slowed reactions times, we also observed that PD subjects took longer to complete the Trailmaking A and B tasks, and this was accompanied by reduced scores on these measures as well (Table 3). For Trailmaking B, this was also true when subjects had to perform the test in a dual task condition, while maintaining upright standing posture (Table 3).

Chemosensory Scores. PD subjects showed highly significant decreased performance in measures of both taste and smell (Table 3). Notably, performance on these two sensory measures was also significantly correlated (Pearson's R=0.27; p=0.015), although this should not be taken as evidence of a strong association between the two measures.

### Microbiome Measures

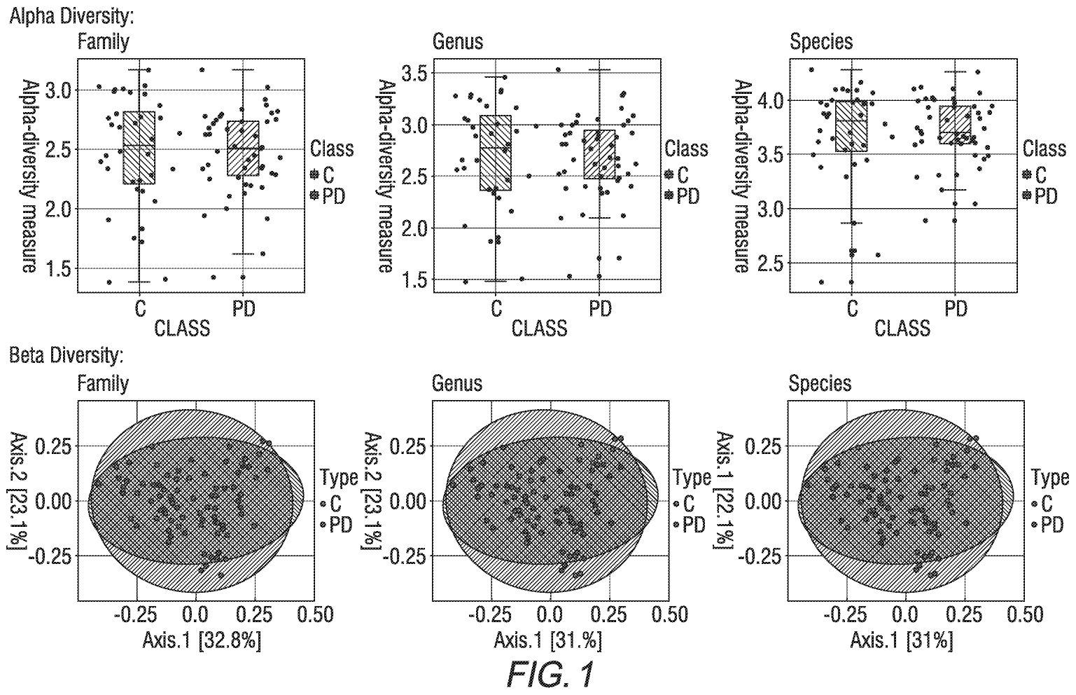

Alpha and Beta Diversity. After filtering to remove taxa that were less consistently observed in the saliva samples, we did not observe any significant differences in overall alpha and beta diversity (FIG. 1) between the two samples. However, it is noteworthy to point out that the PD subjects did appear to show a slightly greater range of beta diversity values when their data were superimposed on those of control subjects.

Genus and Species Differences. A total of 50 microbiome taxa exhibited significant differences in abundance in PD subjects compared with control subjects. These included 16 genera and 34 species, and encompassed bacteria, phage, and Eukaryotic taxa (Table 4) (FDR <0.05). The majority of changes observed were increases in abundance (n=36) rather than decreases in abundance (n=14) (Table 4). Notably, 12 of the genera findings had one or more subordinate species findings, while 4 were changed in isolation. Included among the more commonly changed bacteria species were multiple members of the Lactobacillus (n=6) and Bifidobacterium (n=3) genera (Table 4). We also observed a significant decrease in a bacteriophage (Streptococcus phage Phi Spn 200), and significant increases in three yeast species (Candida albicans, Candida dubliniensis, and Saccharomyces cerevisiae) in PD subjects (Table 4).

TABLE 4
Significantly changed microbiota in early stage PD

| Taxon | Log2 Chg | Std Err | P value | FDR |
|---|---|---|---|---|
| LACTOBACILLUS | 1.61 | 0.35 | 3.31E-06 | 0.000431 |
| Lactobacillus_acidophilus | 2.25 | 0.57 | 7.95E-05 | 0.003984 |
| Lactobacillus_fermenturn | 3.19 | 0.53 | 1.32E-09 | 3.22E-07 |
| Lactobacillus_plantarum | 1.39 | 0.33 | 2.92E-05 | 0.002367 |
| Lactobacillus_reuteri | 1.66 | 0.46 | 0.000283 | 0.010319 |
| Lactobacillus_ruminis | 1.51 | 0.42 | 0.000348 | 0.01207 |
| Lactobacillus_salivarius | 1.15 | 0.36 | 0.001488 | 0.033954 |
| Lutibacter_sp_LP80138 | 1.35 | 0.42 | 0.001143 | 0.028777 |
| METHYLOBACTERIUM | 1.02 | 0.31 | 0.00096 | 0.026802 |
| PARASCARDOVIA | 2.09 | 0.42 | 6.11E-07 | 0.000119 |
| Parascardovia_denticolens | 2.16 | 0.43 | 5.76E-07 | 8.41E-05 |
| RHODOCOCCUS | 0.72 | 0.23 | 0.001425 | 0.032783 |
| Rhodococcus_sp_008 | 1.31 | 0.33 | 8.66E-05 | 0.003984 |
| Saccharomyces_cerevisiae | 1.48 | 0.47 | 0.001479 | 0.033954 |
| SCARDOVIA | 1.41 | 0.40 | 0.000457 | 0.014885 |
| Scardovia_inopinata | 1.43 | 0.41 | 0.000534 | 0.015583 |
| Streptococcus_mutans | 1.43 | 0.41 | 0.000466 | 0.014179 |
| Streptococcus_sp_I_G2 | −1.22 | 0.29 | 2.68E-05 | 0.002367 |
| Streptococcus_phage_PhiSpn_200 | −3.07 | 0.49 | 2.59E-10 | 1.89E-07 |
| TORULASPORA | 1.72 | 0.46 | 0.00019 | 0.007412 |
| Torulaspora_delbrueckii | 1.80 | 0.49 | 0.000228 | 0.00877 |
| WENYINGZHUANGIA | −1.47 | 0.40 | 0.000243 | 0.008622 |
| Wenyingzhuangia_fucanilytica | −1.50 | 0.42 | 0.000364 | 0.01207 |

Significantly changed genera appear in all upper case, with significantly changes species italicized.

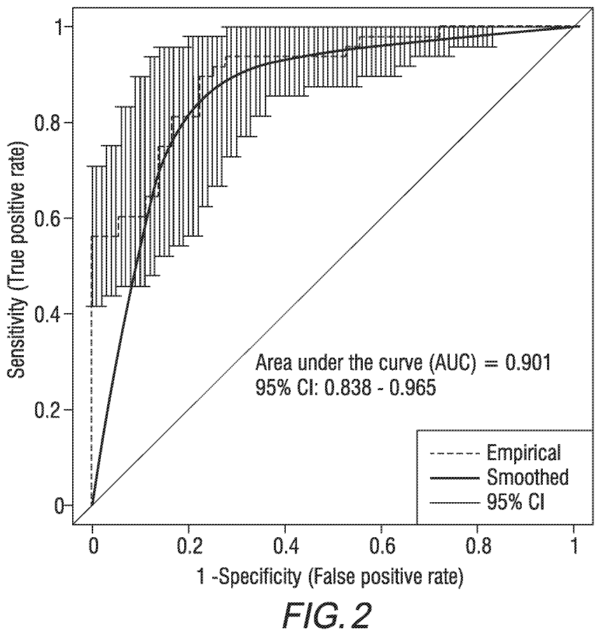

To further probe the consistency of the group microbiome differences, we subjected the genus and species level data to logistic regression classification and area under the receiver operating characteristic curve (ROC) analysis. This indicated a strong separation of the groups was possible using a set of the microbiota data transformed into 5 ratios of two taxa plus 4 additional individual taxa (n=11 taxa total), with an area under the curve (AUC) during training of 0.95 and an AUC during 10-fold cross-validation of 0.90 (FIG. 2). Overall accuracy was 84.5% (13 misclassified subjects out of 84 total).

### Changes in Microbial Transcription Networks in Early Stage PD

In addition to probing for individual transcript alterations, we also tested whether there was evidence of alterations in the expression of microbial metabolic pathways. A total of 167 KEGG pathways were examined, of which 6 showed nominally significant changes (Table 5). The changes included three pathways with increased RNA transcript expression and three with decreased expression.

TABLE 5
Changes in functional curated metabolic pathways in early stage PD.

| KEGG Microbial Pathway | Log2 Chg | P value |
|---|---|---|
| Tryptophan metabolism (ko00380) | −0.718 | 0.0081 |
| Formaldehyde assimilation, serine pathway (M00346) | 0.315 | 0.0120 |
| Citrate cycle TCA cycle, Krebs cycle (M00009) | −0.258 | 0.0385 |
| Citrate cycle TCA cycle (ko00020) | −0.285 | 0.0457 |
| Glycolysis Embden-Meyerhof pathway, | 0.168 | 0.0495 |
| glucose pyruvate (M00001) | | |
| Pentose phosphate pathway | 0.418 | 0.0495 |
| Pentose phosphate cycle (M00004) | | |

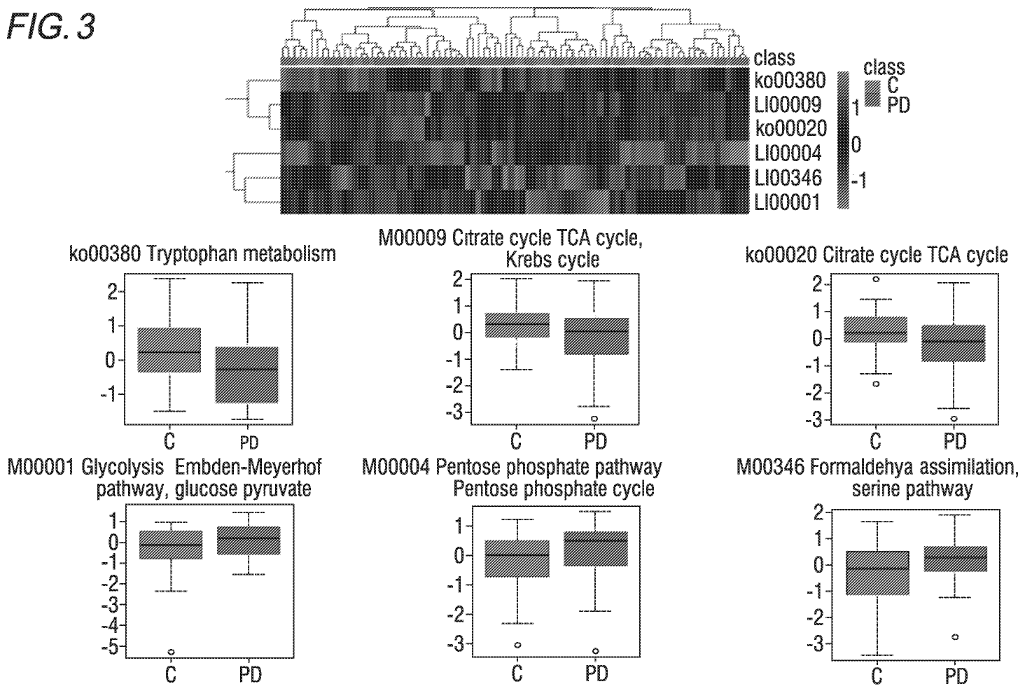

To judge the consistency of microbial metabolic changes in PD subjects, we used hierarchical clustering of the data within the six altered functional pathways and added whisker-box plots (FIG. 3). This indicated relatively consistent separation at the pathway levels. The most visibly shifted pathway based on this analysis was the Tryptophan metabolism pathway (ko00380), which showed a shift of nearly 1 quartile toward decreased expression in PD.

### Correlations of Oral Microbiome and Medical/Demographic Measures

Our final analysis probed for significant associations among the microbial data and the full set of 43 medical, demographic, and functional outcome measures in an exploratory fashion using Pearson correlation analysis. Because of the large number of correlations generated, we used a conservative approach in interpreting the results of these exploratory analyses. Here, we focus only on the 10 most robust correlations in magnitude based on absolute rho value (Table 6). Notably, the magnitude of these correlations all exceeded |0.576| with 9 positive correlations (all at the species level) and a single negative correlation (at the genus level). Interestingly, three of the four most robust correlations were with the Duration of PD (years with diagnosis), while the sole negative correlation was with a balance measure (TSEOFP). As already noted, however, this specific functional task did not show significant differences in performance between PD and control groups (Table 3). Among the 10 most robust overall correlations, only one involved a correlation between a significantly changed taxon (Lactobacillus reuteri) and a significantly changed functional outcome measure (Trailmaking A time to completion).

TABLE 6
Top microbial correlations with medical/demographic/functional measures

| Taxonomy ID: Genus species | R | Subject Measure |
|---|---|---|
| 172045: Elizabethkingia miricola | 0.645 | Duration of PD (years with diagnosis) |
| 526218: Sebaldella termitidis ATCC 33386 | 0.643 | Duration of PD (years with diagnosis) |
| 1112204: Gordonia polyisoprenivorans | 0.624 | Trailmaking B (time) |
| VH2 | | |
| 1408: Bacillus pumilus | 0.618 | Duration of PD (years with diagnosis) |
| 1328: Streptococcus anginosus | 0.604 | Trailmaking B (time) |
| 189423: Streptococcus pneumoniae 670-6B | 0.597 | Trailmaking B Dual Task |
| | | (cognitive score) |
| 242231: Neisseria gonorrhoeae FA 1090 | 0.591 | Trailmaking B (time) |
| 1598: Lactobacillus reuteri* | 0.588 | Trailmaking A (time) |
| 306537: Corynebacterium jeikeium K411 | 0.576 | Trailmaking B (time) |
| 1375: Aerococcus | −0.600 | Tandem Stance Eyes Open Foam Pad |
| | | (sway) |

*showed a significant difference (increased abundance) in PD subjects relative to controls

In another data set, the tope 20 Classifiers in Host and Tax Mixed RNA are shown in Table 7.

TABLE 7
Top 20 Classifiers in Host + Taxa Mixed RNA Models

| Feature | AUC | T-tests | Log2 Chg |
|---|---|---|---|
| hsa-mir-492/hsa-mir-4683 | 0.81655 | 1.95E-07 | −5.2298 |
| 435591 Parabacteroides distasonis | 0.80382 | 4.27E-06 | 6.8823 |
| ATCC 8503/186822 Paenibacillaceae | | | |
| H1FX-AS1/TERC | 0.79977 | 2.19E-05 | 5.0028 |
| hsa-mir-1915/has-mir-4683 | 0.79919 | 2.38E-05 | −4.7229 |
| 1723645 Rhodococcus sp. 008/388396 | 0.7963 | 3.53E-06 | −6.4555 |
| Vibrio fischeri MJ11 | | | |
| ATP2C2-AS1-ZNF337-AS1 | 0.78125 | 1.62E-05 | 4.3306 |
| hsa-mir-130a/hsa-mir-4289 | 0.77894 | 1.12E-05 | 4.6729 |
| FER1L6-AS1/ZFHX4-AS1 | 0.77836 | 3.11E-05 | −4.9958 |
| hsa-miR-183-5p/hsa-miR-149-5p | 0.77778 | 5.78E-05 | −4.1347 |
| 29385 Staphylococcus saprophyticus/1723645 | 0.77431 | 4.64E-06 | 6.0344 |
| Rhodococcus sp. 008 | | | |
| NAPA-AS1/ZNF436-AS1 | 0.77141 | 3.20E-05 | 4.7182 |
| 1790137 Wenyingzhuangia fucanilytica/186822 | 0.77083 | 3.40E-06 | 6.0515 |
| Paenibacillaceae | | | |
| LINC00856/PAN3-AS1 | 0.77025 | 1.35E-05 | −4.6964 |
| 1980001 Cellulosimicrobium sp. TH-20/498214 | 0.76157 | 4.01E-06 | −5.3322 |
| Clostridium botulinum A3 str. Loc Maree | | | |
| hsa-mir-7641-1/hsa-mir-6798 | 0.74942 | 3.48E-05 | −3.7339 |
| hsa-miR-361-3p/hsa-miR-22-5p | 0.74653 | 9.57E-05 | −4.8064 |
| hsa-miR-146a-3p/hsa-miR-338-5p | 0.73322 | 1.59E-04 | −3.8944 |
| hsa-miR-22-5p/hsa-miR-221-5p | 0.73206 | 1.47E-04 | 3.7761 |
| 1723645 Rhodococcus sp. 008 | 0.72975 | 1.34E-04 | −7.1682 |
| piR-hsa-28478/piR-hsa-3405 | 0.72454 | 1.99E-04 | 3.7417 |

The present study defines differences in the oral microbiome in early stage PD as determined from shotgun RNA sequencing of saliva samples combined with detailed phenotypic characterization of subjects. We have six principal findings. First, even in early stage PD, with most subjects on some form of anti-parkinsonian medication, we found evidence of significant (and often highly robust) decreases in balance, sensory, motor and cognitive function. Second, there was no evidence of overall changes in alpha or beta diversity in early stage PD compared with controls. Third, a distinct set of micobial taxa demonstrated consistent changes in sequence abundance at the genus and species level after appropriate correction for multiple testing. Moreover, approximately half of these observed changes fell into clusters of species within the same genera. Fourth, when considered as potential classifiers in a multivariate logistic regression analysis, as few as 11 taxa were found to be capable of distinguishing early stage PD subjects from controls with a 10-fold cross-validated AUC of 0.90 and overall accuracy of 84.5%. Fifth, metabolic pathway analysis of the microbial transcript abundance revealed changes in a distinct subset of biological networks, several of which were highly-related to each other. And sixth, exploratory analyses indicated the presence of highly significant correlations between specific microbiota and specific subsets of functional measures, including a robust correlation between one of the changed microbiota and one of the changed functional measures. In the space that follows, we briefly discuss the importance of these observations.

### Changes in Motor, Cognitive, Balance and Sensory Function in Early Stage PD

Motor impairments are part of the hallmark symptoms of PD, including bradykinesia and rigidity, and represent two of the criteria used in its diagnosis. Thus, the slowing of reaction times and resulting increase in z scores for the speed-based performance elements that we observed are not surprising and are highly-consistent with a vast literature on the topic. Related to this, it is possible that slowing of movements contributed to reduced performance on the specific cognitive outcome measures that we utilized (SRT, PRT, Trailmaking A and B). Notably, however, approximately half the trials on the GNG task actually involve withholding a response, so this task might be expected to be less affected in its overall score than a purely-motor score if the primary issue was motor speed alone. However, our data show highly-similar decreases in both SRT and GNG performance (Table 3), suggesting that the decision-making process does exhibit some impairment as well. The involvement of reduced motor speed in decreased cognitive task performance is further strengthened by examination of the z score magnitudes for the Trailmaking A and B tasks, since the completion times changed more than the scores themselves compared to controls. Again, however, the Trailmaking B showed significant score reductions while the Trailmaking A did not. Thus, although we clearly cannot separate motor and cognitive performance changes in our PD subject cohort, there is a suggestion that the additional cognitive demands of a task result in reduced performance that is added to the effect of bradykinesia.

Another hallmark symptom of PD is postural instability. In the motor examination of the UPDRS, this is usually evaluated through examination of the subjects while standing, walking, turning, and following a pull test. In our cohort of early stage PD subjects, very few individuals exhibited any noticeable impairment in postural stability. Nonetheless, of all the functional measures, the largest z score change in PD subjects was increased body sway compared to controls during a simple static balance task performance (TLEO) (Table 3). This intriguing finding suggests that the computerized functional assessment system we have used to assess PD subjects is highly-sensitive for detecting and quantifying changes in postural sway before they might be obvious or apparent to a trained evaluator.

The final functional domain that we evaluated in our subjects was chemosensory in nature (smell and taste), where our PD cohort scored much worse than the healthy control subjects (Table 3). While not considered pathognomonic, decreased olfaction and taste has been well-documented in PD, including early stages of the disease. Notably, similar decreases in chemosensory function have also been consistently found in subjects with Alzheimer's disease, or a history of mild traumatic brain injury (mTBI). Thus, our findings are consistent with the literature on early stage PD and suggest that these measures may represent useful screening tools, when used in combination with other assessments, for identifying subjects at risk for neurodegenerative disease in general.

Comparison of Microbiome Findings with Prior Studies

Investigations of the GI microbiome has become increasingly prevalent in the past few years, especially in the case of PD which presents with multiple GI symptoms along with motor symptoms and where pathological changes may be occurring well before CNS involvement [see O'Mahony, et al., 2015; Pellegrini et al. 2018]. To date, at least a dozen papers have been published on this topic to probe what might be affected in PD. When the results from these 12 studies are compared, several similar changes can be found, even though they were frequently analyzed at varying levels of classification, and most relied on 16S ribosomal RNA gene sequencing for bacterial identification. Specifically, at the family level of classification, despite differences in tissues and fluids tested, there are overlapping findings from many of the studies, though some bacterial families were less consistent. Eleven of the twelve studies analyzed the microbiomes in fecal stool samples, while one also compared the fecal results to those of sigmoidal colon mucosal biopsies, another compared the fecal results to nasal wash samples, and another investigated potential microbiome changes utilizing oral and nasal swabs (Pereira et al., 2017), although they reported almost no consistent differences in their PD subjects.

Despite the considerable differences in methodology and tissue sources, the results of the present study are highly-consistent with many of those seen in other studies. In fact, half (8/16) of the bacterial families that we found altered were reported to be altered in prior studies (Table 7). In this report, we focus on two of these bacterial families (Bifidobacteriaceae and Lactobacillaceae) which showed similar increases across almost all studies to date and merit further discussion.

Generally regarded as “probiotic” in nature, bacteria within the Bifidobacteriaceae family are proposed to have anti-inflammatory properties and potentially serve beneficial purposes (Mulak and Bonaz., 2015). Thus, it is possible that the changes we and other groups have seen may reflect a compensatory mechanism in the GI tract. However, while Lactobacilli are also generally considered probiotic, some members of the Lactobacillaceae family may exert a disease-worsening effect in PD. Specifically, Lactobacillus reuteri, which we found significantly increased in our PD subjects, was shown in a prior study to increase alpha-synuclein release in the ENS as a result of increasing the firing frequency of mesenteric afferent nerve bundles (by decreasing calcium-dependent potassium channel opening and reducing the slow afterhyperpolarization in these neurons) (Perez-Burgos et al., 2013; Kunze et al., 2009; Paillusson et al., 2012). In this light, it is particularly worthwhile to note that our exploratory correlation analysis identified a robust positive correlation between the abundance of Lactobacillus reuteri and slowing of movement (as reflected in increased performance time on the Trailmaking A test) (Table 6). Other evidence also suggests that Lactobacilli might not be particularly beneficial in PD. Specifically, some members of this bacterial family have been shown to reduce ghrelin secretion, which normally regulates nigrostriatal dopamine and is thought to be neuroprotective, and has been previously reported to be reduced in PD patients (Bayliss et al., 2011; Unger et al., 2011). Thus, based on the available data, the consistent increase in Lactobacillaceae we and others have observed in PD may represent a disadvantageous yet consistent event in the disease. This suggestion lies in stark contrast to much of the current opinion regarding Lactobacilli. Indeed, administration of Lactoballicus reuteri has been shown to reduce anxiety and corticosterone secretion in mice (Bravo et al., 2011), and several other Lactobacillus species have proven beneficial in the treatment of constipation, diarrhea, and IBS symptoms (Fijan, 2014) (see Table 7). Accordingly, we suggest that a closer examination of the benefits and risks of Lactobacillus supplementation is warranted.

Other findings in our PD cohort are worth noting because of their possible relevance to PD and brain function. Among these include changes in several bacterial families that are known to directly affect neurotransmitter levels. These include Lactobacillus and Bifidobacterium genus members already discussed, which produce GABA and acetylcholine (Cryan and Dinan, 2012), Enterobacteriaceae family members, which produce norepinephrine and serotonin and are associated with postural instability and gait difficulty phenotypes in PD (Scheperjans et al., 2015), and members of the Bacillus genus that produce noradrenaline and dopamine (Cryan and Dinan, 2012). Perhaps the most intriguing finding, however, concerns that of the family Nocardiaceae, which includes the Rhodococcus genus and was increased in our early stage PD subjects. The administration of Rhodococcus aurantiacus in laboratory mice was shown to induce encephalitis and cause a movement disorder, due to T-cell mediated inflammation, that subsequently responded in a favorable way to L-DOPA treatment (Min et al., 1999) (Table 7). Thus, the combined set of bacterial families that we observed changed in early stage PD may have broad implications for understanding the pathophysiology of the disorder.

Finally, it is also worthwhile to note that several of the altered microbiota we observed have been linked to PD or are known to play roles in oxidative metabolism. These include members of the Saccharomycetaceae family (encompassing the Candida and Saccaromyces genera), and members of the Acidaminococcaceae and Flavobacteriaceae families (Table 7). Specifically, Candida members produce serotonin and have been anecdotally associated with PD symptoms (Cryan and Dinan, 2012). In contrast, Sacccaromyces cerevisiae produces the rotenone-insensitive NADH:ubiquinone oxidoreductase protein (Ndi1p) which can restore function in complex 1 of the mitochondrial electron transport chain (ETC) that occur due to Pink1 mutations (Vilain et al., 2012). And Acidaminococcus consumes glutamate which is important for oxidation in the intestinal epithelium and is a key contributor to oxidative and amino acid metabolism (Gough et al., 2015). These individual taxon findings are further strengthened by the results of our metabolic pathway findings, which highlighted decreases in Tryptophan and Krebs cycle metabolism and increases in Glycolysis and Pentose phosphate metabolism in early stage PD. Reductions in Tryptophan metabolism could easily lead to reduced serotonin, melatonin and kyenurenate levels, which have all been shown to have neuroprotective properties. And reduced Krebs cycle metabolism could easily lead to overall decreases in ATP production and increased oxidative stress. Viewed this way, the increased Glycolysis and Pentose phosphate pathway activities, could therefore represent compensatory attempts to boost ATP production as well as NADPH levels, with a resulting elevation in reduced glutathione levels leading to greater antioxidant capability. Clearly, further studies are needed to test these suggestions and further characterize the metabolomic profiles of the oral microbiome in early stage PD.

As shown by the results of this study which are disclosed herein, the method according to the invention provides a sensitive, specific, and convenient way to diagnose Parkinson's disease.

### REFERENCES

Parkinson's Disease Information Page (NINDS). http://_www.ninds.nih.gov/Disorders/All-Disorders/Parkinsons-Disease-Information-Last accessed 19 Jan. 2018.Sveinbjornsdottir, S (11 Jul. 2016). “The clinical symptoms of Parkinson's disease”. Journal of Neurochemistry. 139: 318-324. doi:10.1111/jnc.13691. PMID 27401947.
Sveinbjornsdottir, S (11 Jul. 2016). “The clinical symptoms of Parkinson's disease”. Journal of Neurochemistry. 139: 318-324. doi:10.1111/jnc.13691. PMID 27401947abCarroll, William M. (2016). International Neurology. John Wiley & Sons. p. 188. ISBN 9781118777367. Archived from the original on 8 Sep. 2017.
Kalia, L V; Lang, A E (29 Aug. 2015). “Parkinson's disease”. Lancet. 386 (9996): 896-912. doi:10.1016/s0140-6736(14)61393-3. PMID 25904081.
J Ferri, Fred F. (2010). Ferri's differential diagnosis: a practical guide to the differential diagnosis of symptoms, signs, and clinical disorders (2nd ed.). Philadelphia, Pa.: Elsevier/Mosby. p. Chapter P. ISBN 0323076998.\
PLOS ONE 9(8): e106174. https://_doi.org/10.1371/journal.pone.0106174.
Oct. 7, 2015, “Brain-gut-microbiota axis in Parkinson's Disease”. World J. Gastroenterol. V21(37); PMC4588083.
J M Masters, et al., “Elevated salivary protein in Parkinson's disease and salivary DJ-1 as a potential marker of disease severity”. Parkinsonism Relat Disord. 2015 October; 21(10):1251-5. doi: 10.1016/j.parkreldis.2015.07.021. Epub 2015 Jul. 23.
Waddell, P. P., & Salsman, S. P. (2014). Antisense RNA. Salem Press Encyclopedia Of Health,
Bayliss J, Stark R, Reichenbach A, Andrews Z B. Gut hormones restrict neurodegeneration in Parkinson's disease. In: Chang RCC, ed. Advanced Understanding of Neurodegenerative Diseases. 1st ed. New York: Springer, 2011; pp. 269-284.
Unger M M, Moller J C, Mankel K, et al. (2011) Postprandial ghrelin response is reduced in patients with parkinson's disease and idiopathic REM sleep behaviour disorder: a peripheral biomarker for early Parkinson's disease? J Neurol 2011; 258:982-990.
Noyce A J et al (2012) Meta-analysis of early nonmotor features and risk factors for Parkinson disease. Ann Neurol 72:893-901. doi:10. 1002/ana.23687
Van Maele-Fabry G, Hoet P, Vilain F, Lison D (2012) Occupational exposure to pesticides and Parkinson's disease: a systematic review and meta-analysis of cohort studies. Environ Int 46:30-43. doi:10. 1016/j.envint.2012.05.004
Kraus, T. J., Haider, M., Spanner, J., Steinmaurer, M., Dietinger, V., & Kretzschmar, H. A. (2017). Altered Long Noncoding RNA Expression Precedes the Course of Parkinson's Disease—a Preliminary Report. Molecular Neurobiology, (4), 2869. doi:10.1007/s12035-016-9854-x
Yang, L., Froberg, J. E., Lee, J. T. (2014) Long noncoding RNAs: fresh perspectives into the RNA world. Trends in Biochemical Sciences, 39 (1), 35-43. https://doi.org/10.1016/j.tibs.2013.10.002
Liu, Y., Fallon, L., Lashuel, H. A., Liu, Z., and Lansbury, P. T. Jr. (2002). The UCH-L1 gene encodes two opposing enzymatic activities that affect alpha-synuclein degradation and Parkinson's disease susceptibility. Cell 111, 209-218. doi: 10.1016/S0092-8674(02)01012-7
Choi, J., Levey, A. I., Weintraub, S. T., Rees, H. D., Gearing, M., Chin, L. S., et al. (2004). Oxidative modifications and down-regulation of ubiquitin carboxyl-terminal hydrolase L1 associated with idiopathic Parkinson's and Alzheimer's diseases. J Biol. Chem. 279, 13256-13264. doi: 10.1074/jbc.M314124200
Barrachina, M., Castano, E., Dalfo, E., Maes, T., Buesa, C., and Ferrer, I. (2006). Reduced ubiquitin C-terminal hydrolase-1 expression levels in dementia with Lewy bodies. Neurobiol. Dis. 22, 265-273. doi: 10.1016/j.nbd.2005.11.005
Soreq L, Guffanti A, Salomonis N, Simchovitz A, Israel Z, et al. (2014) Long Non-Coding RNA and Alternative Splicing Modulations in Parkinson's Leukocytes Identified by RNA Sequencing. PLOS Computational Biology 10(3): e1003517. https://doi.org/10.1371/j ournal.pcbi.1003517
Majidinia, M., Mihanfar, A., Rahbarghazi, R., Nourazarian, A., Bagca, B., & Avci, C. B. (2016). The roles of non-coding RNAs in Parkinson's disease. Molecular Biology Reports, (11), 1193. doi:10.1007/s11033-016-4054-3
Azucena Perez-Burgos, Bingxian Wang, Yu-Kang Mao, Bhavik Mistry, Karen-Anne McVey Neufeld, John Bienenstock, and Wolfgang Kunze. (2013). Psychoactive bacteria Lactobacillus rhamnosus (JB-1) elicits rapid frequency facilitation in vagal afferents. American Journal of Physiology-Gastrointestinal and Liver Physiology 2013 304:2, G211-G220
Kunze, W. A., Mao, Y., Wang, B., Huizinga, J. D., Ma, X., Forsythe, P. and Bienenstock, J. (2009), Lactobacillus reuteri enhances excitability of colonic AH neurons by inhibiting calcium-dependent potassium channel opening. Journal of Cellular and Molecular Medicine, 13: 2261-2270. doi:10.1111/j.1582-4934.2009.00686.x
Sébastien Paillusson, Thomas Clairembault, Mandy Biraud, Michel Neunlist, Pascal Derkinderen (2012). Activity-dependent secretion of alpha-synuclein by enteric neurons. Journal of Neurochemistry. 125: 512-517. doi: 10.1111/jnc.12131
Scheperjans, F., Aho, V., Pereira, P. A., Koskinen, K., Paulin, L., Pekkonen, E., Haapaniemi, E., Kaakkola, S., Eerola-Rautio, J., Pohja, M., Kinnunen, E., Murros, K. and Auvinen, P. (2015), Gut microbiota are related to Parkinson's disease and clinical phenotype. Mov Disord., 30: 350-358. doi:10.1002/mds.26069\
Hill Burns, E. M., Debelius, J. W., Morton, J. T., Wissemann, W. T., Lewis, M. R., Wallen, Z. D., Peddada, S. D., Factor, S. A., Molho, E., Zabetian, C. P., Knight, R. and Payami, H. (2017), Parkinson's disease and Parkinson's disease medications have distinct signatures of the gut microbiome. Mov Disord., 32: 739-749. doi:10.1002/mds.26942
Keshavarzian, A., Green, S. J., Engen, P. A., Voigt, R. M., Naqib, A., Forsyth, C. B., Mutlu, E. and Shannon, K. M. (2015), Colonic bacterial composition in Parkinson's disease. Mov Disord., 30: 1351-1360. doi:10.1002/mds.26307
Hasegawa, S., Goto, S., Tsuji, H., Okuno, T., Asahara, T., Nomoto, K., . . . Hirayama, M. (2015). Intestinal Dysbiosis and Lowered Serum Lipopolysaccharide-Binding Protein in Parkinson's Disease. PLoS ONE, 10(11), e0142164. http://doi.org/10.1371/journal.pone.0142164
Unger, M. M., . . . & Schafer K. (2016). Short chain fatty acids and gut microbiota differ between patients with Parkinson's disease and age-matched controls. Parkinsonism & Related Disorders, 32. 66-72.
Bedarf, J. R., . . . & Wullner, U. (2017) Functional Implications of Microbal and Viral Gut Metagenome Changes in Early Stage L-DOPA-naïve Parkinson's Disease Patients. Genome Medicine, 9(39) DOI 10.1186/s13073-017-0428-y
Qian, Y., Yang, X., Xu, S., Wu, C., Song, Y., Qin, N., & Xiao, Q. (2018). Full-length Article: Alteration of the fecal microbiota in Chinese patients with Parkinson's disease. Brain Behavior And Immunity, 70194-202. doi:10.1016/j.bbi.2018.02.016
Hopfner, F., Kiinstner, A., Müller, S. H., Kiinzel, S., Zeuner, K. E., Margraf, N. G., & Kuhlenbäumer, G. (2017). Research report: Gut microbiota in Parkinson disease in a northern German cohort. Brain Research, 166741-45. doi:10.1016/j.brainres.2017.04.019
Pereira, P. B., Aho, V. E., Paulin, L., Pekkonen, E., Auvinen, P., & Scheperjans, F. (2017). Oral and nasal microbiota in Parkinson's disease. Parkinsonism & Related Disorders, 3861-67. doi:10.1016/j.parkreldis.2017.02.026
Petrov, V. A., Saltykova, I. V., Zhukova, I. A., Alifirova, V. M., Zhukova, N. G., Dorofeeva, Y. B., & . . . Sazonov, A. E. (2017). Analysis of Gut Microbiota in Patients with Parkinson's Disease. Bulletin Of Experimental Biology And Medicine, 162(6), 734-737. doi:10.1007/s10517-017-3700-7
Faghihi M A, Wahlestedt C (2009) Regulatory roles of natural antisense transcripts. Nat Rev Mol Cell Biol 10(9):637-643
Ventura M, Turroni F, Lugli G A, van Sinderen D. Bifidobacteria and humans: our special friends, from ecological to genomics perspectives. J Sci Food Agric. 2014; 94(2):163-8. pmid:23963950
Alton G G, Forsyth J R L. Brucella. In: Baron S, editor. Medical Microbiology. 4th edition. Galveston (Tex.): University of Texas Medical Branch at Galveston; 1996. Chapter 28. Available from: https://www.ncbi.nih.gov/books/NBK8572/
Loesche W. J. Role of Streptococcus mutans in human dental decay. Microbiol. Mol. Biol. Rev. 1986; 50:353-380.
Guttman, M., & Rinn, J. L. (2012). Modular regulatory principles of large non-coding RNAs. Nature, 482(7385), 339-346. http://doi.org/10.1038/nature10887
Lu, Z., Carter, A. C., & Chang, H. Y. (2017). Mechanistic insights in X-chromosome inactivation. Philosophical Transactions Of The Royal Society B-Biological Sciences, 372(1733),
Qureshi, I. A., & Mehler, M. F. (2012). Emerging roles of non-coding RNAs in brain evolution, development, plasticity and disease. Nature Reviews. Neuroscience, 13(8), 528-541. http://doi.org/10.1038/nrn3234
Cao X, Yeo G, Muotri A R, Kuwabara T, Gage F H (2006) Noncoding RNAs in the mammalian central nervous system. Annu Rev Neurosci 29: 77-103.
Matouk I J, DeGroot N, Mezan S, Ayesh S, Abu-lail R, et al. (2007) The H19 non-coding RNA is essential for human tumor growth. PLoS One 2: e845.
Cryan, J. F., & Dinan, T. G. (2012). Mind-altering microorganisms: the impact of the gut microbiota on brain and behaviour. Nature Reviews Neuroscience, 13(10), 701-712.
Messaoudi, M. et al. Assessment of psychotropic-like properties of a probiotic formulation (Lactobacillus helveticus R0052 and Bifidobacterium longum R0175) in rats and human subjects. Br. J. Nutr. 105, 755-764 (2011).
Bravo, J. A. et al. Ingestion of Lactobacillus strain regulates emotional behavior and central GABA receptor expression in a mouse via the vagus nerve. Proc. Natl Acad. Sci. USA 108, 16050-16055 (2011)
Heintz-Buschart, A., Pandey, U., Wicke, T., Sixel-Doring, F., Janzen, A., Sittig-Wiegand, E., Trenkwalder, C., Oertel, W. H., Mollenhauer, B. and Wilmes, P. (2018), The nasal and gut microbiome in Parkinson's disease and idiopathic rapid eye movement sleep behavior disorder. Mov Disord., 33: 88-98. doi:10.1002/mds.27105
Li, W., Wu, X., Hu, X. et al. Structural changes of gut microbiota in Parkinson's disease and its correlation with clinical features. Sci. China Life Sci. (2017) 60: 1223. https://doi.org/10.1007/s11427-016-9001-4.
Fijan, S. (2014). Microorganisms with Claimed Probiotic Properties: An Overview of Recent Literature. International Journal of Environmental Research and Public Health, 11(5), 4745-4767. http://doi.org/10.3390/ijerph110504745
Choi, D. Y., & Choi, H. (2015). Natural products from marine organisms with neuroprotective activity in the experimental models of Alzheimer's disease, Parkinson's disease and ischemic brain stroke: their molecular targets and action mechanisms. ARCHIVES OF PHARMACAL RESEARCH, (2). 139.
Min, Y., Asano, M., Kohanawa, M., & Minagawa, T. (1999). Movement disorders in encephalitis induced by Rhodococcus aurantiacus infection relieved by the administration of L-dopa and anti-T-cell antibodies. Immunology, 96(1), 10-15. http://doi.org/10.1046/j.1365-2567.1999.00659.x
Gough, E. K., Stephens, D. A., Moodie, E. E. M., Prendergast, A. J., Stoltzfus, R. J., Humphrey, J. H., & Manges, A. R. (2015). Linear growth faltering in infants is associated with Acidaminococcus sp. and community-level changes in the gut microbiota. Microbiome, 3, 24. http://doi.org/10.1186/s40168-015-0089-2
Yu, S., Zhou, X., Li, F., Xu, C., Zheng, F., Li, J., . . . Feng, Y. (2017). Microbial transformation of ginsenoside Rb1, Re and Rg1 and its contribution to the improved anti-inflammatory activity of ginseng. Scientific Reports, 7, 138. http://doi.org/10.1038/s41598-017-00262-0
Mulak, A., & Bonaz, B. (2015). Brain-gut-microbiota axis in Parkinson's disease. World Journal of Gastroenterology: WJG, 21(37), 10609-10620. http://doi.org/10.3748/wjg.v21.i37.10609
Lastovica A. J., On S. L. W., Zhang L. (2014) The Family Campylobacteraceae. In: Rosenberg E., DeLong E. F., Lory S., Stackebrandt E., Thompson F. (eds) The Prokaryotes. Springer, Berlin, Heidelberg. https://doi.org/10.1007/978-3-642-39044-9_274
Chang, Q., Ling, Z., Paul M, C., & Jack W, L. (2003). Prenatal Exposure to the Bacteriotoxin Lipopolysaccharide Leads to Long-Term Losses of Dopamine Neurons in Offspring: A Potential, New Model of Parkinson's Disease. Frontiers In Bioscience, 8(1), s826.
Oshima, K., Hayashi, J., Toh, H., Nakano, A., Shindo, C., Komiya, K., . . . Hattori, M. (2015). Complete Genome Sequence of Parascardovia denticolens JCM 12538T, Isolated from Human Dental Caries. Genome Announcements, 3(3), e00485-15.
http://doi.org/10.1128/genomeA.00485-15 Vilain, S., Esposito, G., Haddad, D., Schaap, O., Dobreva, M. P., Vos, M., & . . . Verstreken, P. (2012). The Yeast Complex I Equivalent NADH Dehydrogenase Rescues pinkl Mutants. Plos Genetics, 8(1), 1-13. doi:10.1371/journal.pgen.1002456
Surwase, S. N. & Jadhav, J. P. Bioconversion of 1-tyrosine to 1-DOPA by a novel bacterium Bacillus sp. JPJ Amino Acids (2011) 41: 495. https://doi.org/10.1007/s00726-010-0768-z
Frost F and Nazareth D. Case Report: First report of Elizabethkingia miricola infection in a patient with cystic fibrosis. F1000Research 2018, 7:440

All publications and patent applications mentioned in this specification are herein incorporated by reference in their entirety to the same extent as if each individual publication or patent application was specifically and individually indicated to be incorporated by reference, especially referenced is disclosure appearing in the same sentence, paragraph, page or section of the specification in which the incorporation by reference appears.

The citation of references herein does not constitute an admission that those references are prior art or have any relevance to the patentability of the technology disclosed herein. Any discussion of the content of references cited is intended merely to provide a general summary of assertions made by the authors of the references, and does not constitute an admission as to the accuracy of the content of such references.

1. A method for detecting a risk of Parkinson's Disease (“PD”) comprising:
detecting one or more micro-RNAs and/or microbial RNAs associated with PD in saliva of a subject, and
detecting a risk of PD when said microRNA and/or microbial RNA is present in an amount significantly below or above that detected in a control subject; and optionally, when an abnormal lower or higher level is detected, further evaluating the patient for Parkinson's Disease or treating the subject for Parkinson's Disease.

2. The method of claim 1, wherein detecting comprises detecting an abnormal level of one or more miRNAs and/or microbial RNAs associated with one or more Parkinson's symptoms, ratings or scores selected from the group consisting of UPDRS-1 rating, UPDRS-II rating, UPDRS-III rating, duration of PD, resting tremor, anti-PD medication, sleep dysfunction, oropharyngeal dysfunction, thermoregulatory dysfunction, vasomotor dysfunction, gastrointestinal dysfunction, urinary dysfunction, NMS questionnaire evaluation or rating, SCOPA-AUT evaluation or rating, PDQUALIF scale rating, Beck Depression inventory rating, and one or more functional outcome measures.

3. The method of claim 1, wherein detecting comprises detecting at least one miRNA and at least one microbial RNA.

4. The method of claim 1, wherein the detecting detects at least one host+taxa classifier selected from the group consisting of hsa-mir-492/hsa-mir-4683, 435591 Parabacteroides distasonis ATCC 8503/186822 Paenibacillaceae, H1FX-AS1/TERC, hsa-mir-1915/hsa-mir-4683, 1723645 Rhodococcus sp. 008/388396 Vibrio fischeri MJ11, ATP2C2-AS1/ZNF337-AS1, hsa-mir-130a/hsa-mir-4289, FER1L6-AS1/ZFHX4-AS1, hsa-miR-183-5p/hsa-miR-149-5p, 29385 Staphylococcus saprophyticus/1723645 Rhodococcus sp. 008, NAPA-AS1/ZNF436-AS1, 1790137 Wenyingzhuangia fucanilytica/186822 Paenibacillaceae, LINC00856/PAN3-AS1, 1980001 Cellulosimicrobium sp. TH-20/498214 Clostridium botulinum A3 str. Loch Maree, hsa-mir-7641-1/hsa-mir-6798, hsa-miR-361-3p/hsa-miR-22-5p, hsa-miR-146a-3p/hsa-miR-338-5p, hsa-miR-22-5p/hsa-miR-221-5p, 1723645 Rhodococcus sp. 008, and piR-hsa-28478/piR-hsa-3405.

5. The method of claim 4, wherein the detecting detects at least 2, 3, 4, 5, 6, 7, 8, 9, 10, 11, 12, 13, 14, 15, 16, 17, 18, 19, or 20 classifiers.

6. The method of claim 1, wherein detecting comprises detecting at least two classifiers selected from the group consisting of hsa-mir-18915/hsa-mir, H1FX-AS1/TERC, 4235591 Parabaceroid, has-mir-492-hsa-mir, R1L6-AS1/ZFHX4-AS1, LINC00856/PAN3-AS1, 1980001 Cellulosimic, 723645 Rhodococcus, hsa-miR-183-5p/has-m, has-mir-130a/hsa-mir, has-mir-7641-1/hsa-m, 790137 Wenyingzhuan, 33014 Clavibacter mi, 723645 Rhodococcus, 29385 Staphylococcus, hsa-miR-361-3p/hsa-m, APA-AS1/ZNF337-AS1, P2C2-AS1/ZNF337-AS, pir-has-28478/piR-hs, hsa-miR-146a-3p/has, has-miR-22-5p/has-mi, pir-has-5937/piR-hsa, pir-hsa-12487/piR-hs, hsa-miR-146a-3p/hsa, piR-hsa-5937/piR-hsa, piR-hsa-28875/piR-hs, hsa-miR-361-3p, 1598 Lactobacillus r, 991789 Clostridium p, hsa-miR-22-5p, and hsa-miR-221-5p.

7. The method of claim 6, wherein the detecting detects at least 3, 4, 5, 6, 7, 8, 9, 10, 11, 12, 13, 14, 15, 16, 17, 18, 19, 20, 21, 22, 23, 24, 25, 26, 27, 28, 29, 30 or 31 classifiers.

8. The method of claim 1 that detects a subject with PD with an accuracy of at least 90%.

9. The method of claim 1 that further comprises monitoring the levels of one or more miRNAs or microbial RNAs as an index of progression or regression of PD.

10. The method of claim 1 that further comprises treating a subject for PD and monitoring the levels of one or more miRNAs or microbial RNAs as an index of progression or regression of PD before, during or after treatment.

11. A composition comprising probes and or primers that identify at least one salivary miRNA or microbial RNA associated with PD.

12. The composition of claim 11, wherein the probes and/or primers identify at least 2, 3, 4, 5, 6, 7, 8, 9, 10, 15, 20, 50 or more miRNAs or microbial RNAs.

13. The composition of claim 11 that comprises probes and/or primers that identify at least one host+taxa classifier selected from the group consisting of hsa-mir-492/hsa-mir-4683, 435591 Parabacteroides distasonis ATCC 8503/186822 Paenibacillaceae, H1FX-AS1/TERC, hsa-mir-1915/hsa-mir-4683, 1723645 Rhodococcus sp. 008/388396 Vibrio fischeri MJ11, ATP2C2-AS1/ZNF337-AS1, hsa-mir-130a/hsa-mir-4289, FER1L6-AS1/ZFHX4-AS1, hsa-miR-183-5p/hsa-miR-149-5p, 29385 Staphylococcus saprophyticus/1723645 Rhodococcus sp. 008, NAPA-AS1/ZNF436-AS1, 1790137 Wenyingzhuangia fucanilytica/186822 Paenibacillaceae, LINC00856/PAN3-AS1, 1980001 Cellulosimicrobium sp. TH-20/498214 Clostridium botulinum A3 str. Loch Maree, hsa-mir-7641-1/hsa-mir-6798, hsa-miR-361-3p/hsa-miR-22-5p, hsa-miR-146a-3p/hsa-miR-338-5p, hsa-miR-22-5p/hsa-miR-221-5p, 1723645 Rhodococcus sp. 008, and piR-hsa-28478/piR-hsa-3405.

14. The composition of claim 11 that comprises probes and/or primers that identify at least one host+taxa classifier selected from the group consisting of hsa-mir-18915/hsa-mir, H1FX-AS1/TERC, 4235591 Parabaceroid, has-mir-492-hsa-mir, R1L6-AS1/ZFHX4-AS1, LINC00856/PAN3-AS1, 1980001 Cellulosimic, 723645 Rhodococcus, hsa-miR-183-5p/has-m, has-mir-130a/hsa-mir, has-mir-7641-1/hsa-m, 790137 Wenyingzhuan, 33014 Clavibacter mi, 723645 Rhodococcus, 29385 Staphylococcus, hsa-miR-361-3p/hsa-m, APA-AS1/ZNF337-AS1, P2C2-AS1/ZNF337-AS, pir-has-28478/piR-hs, hsa-miR-146a-3p/has, has-miR-22-5p/has-mi, pir-has-5937/piR-hsa, pir-hsa-12487/piR-hs, hsa-miR-146a-3p/hsa, piR-hsa-5937/piR-hsa, piR-hsa-28875/piR-hs, hsa-miR-361-3p, 1598 Lactobacillus r, 991789 Clostridium p, hsa-miR-22-5p, and hsa-miR-221-5p.

15. The composition of claim 11 that is a microarray, biochip or chip.

16. A system for detecting miRNA and microbial RNA in saliva comprising a microarray containing probes or primers according to claim 11 that recognize multiple miRNA and microbial RNAs associated with Parkinson's Disease, and optionally signal transmission, information processing, and data display or output elements

17. The system of claim 16, further comprising one or more elements for receiving, and optionally purifying or isolating miRNA and/or microbial RNA.

18. A composition comprising one or more miRNAs according to claim 11 the levels of which are lower than in a healthy control who does not have PD, in a form suitable for administration to a tissue or site affected by Parkinson's disease.

19. The composition of claim 18 in a form of a natural or synthetic liposome, microvesicle, protein complex, lipoprotein complex, exosome or multivesicular body; or probiotic or prebiotic product.

20. A method for treating a subject at risk of Parkinson's disease, or having Parkinson's disease, comprising administering the composition of claim 18 to a subject in need thereof.

21. (canceled)

22. (canceled)
